(19) United States
(12) Patent Application Publication
(10) Pub. No.: US 2016/0143883 A1
(43) Pub. Date: May 26, 2016

(54) METHOD OF TREATING THROMBOCYTOPENIA

(71) Applicants: Shivakumar G. Kapsi, Collegeville, PA (US); Francis Xavier Muller, Collegeville, PA (US); Mary Beth Wire, Research Triangle Park, NC (US)

(72) Inventors: Shivakumar G. Kapsi, Collegeville, PA (US); Francis Xavier Muller, Collegeville, PA (US); Mary Beth Wire, Research Triangle Park, NC (US)

(73) Assignee: NOVARTIS AG, Basel (CH)

(21) Appl. No.: 14/975,930

(22) Filed: Dec. 21, 2015

Related U.S. Application Data
(63) Continuation of application No. 14/150,112, filed on Jan. 8, 2014.
Application No. 14/150,112 is a continuation of application No. 13/124,182, filed on Aug. 9, 2011, now abandoned.
(60) Provisional application No. 61/105,927, filed on Oct. 16, 2008.

(51) Int. Cl. A61K 31/415

(52) U.S. Cl. CPC A61K 31/415

(57) ABSTRACT

Invented is a method of treating thrombocytopenia in a human in need thereof which comprises the administration of a therapeutically effective amount of a 3′-{N′-[1-(3,4-Dimethylphenyl)-3-methyl-5-oxo-1,5-dihydropyrazol-4-ylidene]hydrazino}-2′-hydroxybiphenyl-3-carboxylic acid to such human.

### FIELD OF THE INVENTION

This invention relates to a method of treating thrombocytopenia in a human by the in vivo administration of 3′-[(2Z)-[1-(3,4-dimethylphenyl)-1,5-dihydro-3-methyl-5-oxo-4H-pyrazol-4-ylidene]hydrazino]-2′-hydroxy-[1,1′-biphenyl]-3-carboxylic acid or a pharmaceutically acceptable salt thereof, suitably the bis-(monoethanolamine) salt, (hereinafter the bis-(monoethanolamine) salt is Compound A;

which is a compound is represented by Structure I:

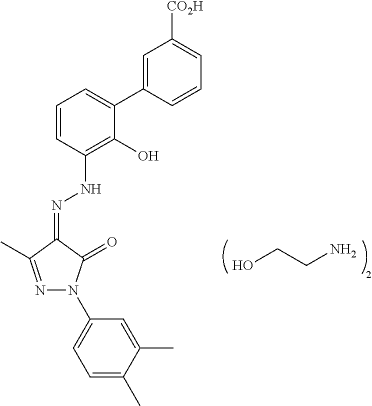

and the corresponding salt free compound is Compound B).

### BACKGROUND OF THE INVENTION

Thrombopoietin (TPO) has been shown to be the main humoral regulator in situations involving thrombocytopenia. See, e.g., Metcalf Nature 369:519-520 (1994). TPO has been shown in several studies to increase platelet counts, increase platelet size, and increase isotope incorporation into platelets of recipient animals. Because platelets (thrombocytes) are necessary for blood clotting and when their numbers are very low a patient is at risk of death from catastrophic hemorrhage, TPO is considered to have potential useful applications in both the diagnosis and the treatment of various hematological disorders, for example, diseases primarily due to platelet defects. In addition, studies have provided a basis for the projection of efficacy of TPO therapy in the treatment of thrombocytopenia, and particularly thrombocytopenia resulting from chemotherapy, radiation therapy, or bone marrow transplantation as treatment for cancer or lymphoma. See e.g., McDonald (1992) Am. J. Ped. Hematology/Oncology 14: 8-21 (1992).

The slow recovery of platelet levels in patients suffering from thrombocytopenia is a serious problem, and has lead to the search for small molecule non-peptide TPO receptor agonists that are able to accelerate platelet regeneration. (e.g. see, International Application Number PCT/US01/16863, having International Filing Date May 24, 2001, which specifically discloses Compound B, in Example 3, and the use of non-peptide TPO receptor agonists in combination with further active ingredients). International Application Number PCT/US01/16863 specifically includes the treatment of thrombocytopenia wherein the thrombocytopenia is due to: myelosuppression, organ transplant, bone marrow transplant, stem cell transplant, liver transplant, idiopathic thrombocytopenia purpura (ITP), myelodysplastic syndromes (MDS), aplastic anemia. leukemia, viral infection, fungal infection, microbial infection. parasitic infection, liver dysfunction, surgical procedures, treatment with antiviral agents, and treatment with antibiotic agents.

Compound A is disclosed in International Application No. PCT/US03/16255, having an International filing date of May 21, 2003; International Publication Number WO 03/098002 and an International Publication date of Dec. 4, 2003.

Non-peptide TPO receptor agonists, including Compound A, are disclosed for the treatment of degenerative diseases/injuries in International Application No. PCT/US04/013468, having an International filing date of Apr. 29, 2004; International Publication Number WO 04/096154 and an International Publication date of Nov. 11, 2004.

It would be advantageous to provide an improved method of treating thrombocytopenia.

It would be advantageous to provide an improved method of administering Compound A and Compound B.

### SUMMARY OF THE INVENTION

This invention comprises a method of administering Compound A, or Compound B or a pharmaceutically acceptable salt thereof, which method takes into account adverse reactions of the compound.

This invention comprises a method of administering Compound A, or Compound B or a pharmaceutically acceptable salt thereof, which method takes into account the toxicology of the compound.

This invention comprises a method of administering Compound A, or Compound B or a pharmaceutically acceptable salt thereof, which method takes into account the risk for hepatotoxicity of the compound.

This invention comprises a method of administering Compound A, or Compound B or a pharmaceutically acceptable salt thereof, which method takes into account use in specific populations for the compound.

This invention comprises a method of administering Compound A, or Compound B or a pharmaceutically acceptable salt thereof, which method takes into account thrombotic/thromboembolic complications of the compound.

This invention comprises a method of administering Compound A, or Compound B or a pharmaceutically acceptable salt thereof, which method takes into account the clinical pharmacology of the compound.

This invention comprises a method of administering Compound A, or Compound B or a pharmaceutically acceptable salt thereof, which method takes into account Drug Interactions of the compound.

### DETAILED DESCRIPTION OF THE INVENTION

FULL PRESCRIBING INFORMATION
WARNING: Risk for Hepatotoxicity

```
Promacta ® may cause hepatotoxicity:
  Measure serum alanine aminotransferase (ALT), aspartate aminotransferase (AST), and
  bilirubin prior to initiation of Promacta ®, every 2 weeks during the dose adjustment
  phase and monthly following establishment of a stable dose. If bilirubin is elevated,
  perform fractionation.
  Evaluate abnormal serum liver tests with repeat testing within 3 to 5 days. If the
  abnormalities are confirmed, monitor serum liver tests weekly until the
  abnormality(ies) resolve, stabilize or return to baseline levels.
  Discontinue Promacta ® if ALT levels increase to ≧3X the upper limit of normal [ULN]
  and are:
      progressive, or
      persistent for ≧4 weeks, or
      accompanied by increased direct bilirubin, or
      accompanied by clinical symptoms of liver injury or evidence for hepatic
      decompensation.
Because of the risk for hepatotoxicity and other risks [seeWarnings and Precautions (5.1-5.6)]
Promacta ® is available only through a restricted distribution program called
PROMACTA ® CARES. Under PROMACTA ® CARES, only prescribers, pharmacies and
patients registered with the program are able to prescribe, dispense and receive Promacta ®.
To enroll in PROMACTA ® CARES, call 1-877-9-PROMACTA ® [seeWarnings and
Precautions (5.8)].
```

### 1 Indications and Usage

Promacta® is indicated for the treatment of thrombocytopenia in patients with chronic immune (idiopathic) thrombocytopenic purpura (ITP) who have had an insufficient response to corticosteroids, immunoglobulins, or splenectomy. Promacta® should be used only in patients with ITP whose degree of thrombocytopenia and clinical condition increases the risk for bleeding. Promacta® should not be used in an attempt to normalize platelet counts.

### 2 Dosage and Administration

Only prescribers enrolled in PROMACTA® CARES may prescribe Promacta® [see Warnings and Precautions (5.8)].

Monitor liver tests (ALT, AST and bilirubin) and complete blood counts (CBCs), including platelet counts and peripheral blood smears, prior to initiation of Promacta® and throughout therapy with Promacta®. If bilirubin is elevated, perform fractionation. Monitor CBCs, including platelet counts, for at least 4 weeks following discontinuation of Promacta® [see Warnings and Precautions (5.3)]. In clinical studies, platelet counts generally increased within 1 to 2 weeks after starting Promacta® and decreased within 1 to 2 weeks after discontinuing Promacta® [see Clinical Studies (14)].

Use the lowest dose of Promacta® to achieve and maintain a platelet count≧50×10⁹/L as necessary to reduce the risk for bleeding. Dose adjustments are based upon the platelet count response. Do not use Promacta® in an attempt to normalize platelet counts [see Warnings and Precautions (5.4)].

Take Promacta® on an empty stomach (1 hour before or 2 hours after a meal [see Pharmacokinetics (12.3)]. Allow at least a 4-hour interval between Promacta® and other medications (e.g., antacids), calcium-rich foods (e.g., dairy products and calcium fortified juices), or supplements containing polyvalent cations such as iron, calcium, aluminum, magnesium, selenium, and zinc [see Drug Interactions (7.4) and Clinical Pharmacology (12.3)].

### 2.1 Initial Dose Regimen:

Initiate Promacta® at a dose of 50 mg once daily except in patients who are of East Asian ancestry or who have moderate to severe hepatic impairment.

For patients of East Asian ancestry (such as Chinese, Japanese, Taiwanese, or Korean), initiate Promacta® at a reduced dose of 25 mg once daily [see Clinical Pharmacology (12.3)].

For patients with moderate or severe hepatic impairment, initiate Promacta® at a reduced dose of 25 mg once daily [see Use in Specific Populations (8.6)].

### 2.2 Monitoring and Dose Adjustment

After initiating Promacta®, adjust the dose to achieve and maintain a platelet count≧50×10⁹/L as necessary to reduce the risk for bleeding. Do not exceed a dose of 75 mg daily. Monitor clinical hematology and liver tests regularly throughout therapy with Promacta® and modify the dosage regimen of Promacta® based on platelet counts as outlined in Table 1. During therapy with Promacta®, assess CBCs, including platelet count and peripheral blood smears, weekly until a stable platelet count has been achieved. Obtain CBCs including platelet counts and peripheral blood smears, monthly thereafter.

TABLE 1
Dose Adjustments of Promacta ®

| Platelet Count Result | Dose Adjustment or Response |
|---|---|
| <50 × 10⁹/L following at least 2 | Increase daily dose by 25 mg to a maximum of 75 mg daily; |
| weeks of Promacta ® | |
| ≧200 × 10⁹/L to ≦400 × 10⁹/L at | Decrease the daily dose by 25 mg. Wait 2 weeks to assess |
| any time | the effects of this and any subsequent dose adjustments. |
| >400 × 10⁹/L | Stop Promacta ®; increase the frequency of platelet |
| | monitoring to twice weekly. Once the platelet count is <150 × |
| | 10⁹/L reinitiate therapy at a daily dose reduced by 25 mg. |
| >400 × 10⁹/L after 2 weeks of | Permanently discontinue Promacta ®. |
| therapy at lowest dose of | |
| Promacta ® | |

Modify the dosage regimen of concomitant TTP medications, as medically appropriate, to avoid excessive increases in platelet counts during therapy with Promacta®. Do not administer more than one dose of Promacta® within any 24-hour period.

### 2.3 Discontinuation:

Discontinue Promacta® if the platelet count does not increase to a level sufficient to avoid clinically important bleeding after 4 weeks of therapy with Promacta® at the maximum daily dose of 75 mg. Excessive platelet count responses, as outlined in Table 1, or important liver test abnormalities also necessitate discontinuation of Promacta® [see Warnings and Precautions (5.1)].

### 3 Dosage Forms and Strengths

25 mg tablets—round, biconvex, orange, film-coated tablets debossed with GS NX3 and 25 on one side. Each tablet, for oral administration, contains eltrombopag olamine, equivalent to 25 mg of eltrombopag free acid.

50 mg tablets—round, biconvex, blue, film-coated tablets debossed with GS UFU and 50 on one side. Each tablet, for oral administration, contains eltrombopag olamine, equivalent to 50 mg of eltrombopag free acid.

### 4 Contraindications

None.

### 5 Warnings and Precautions

### 5.1 Risk for Hepatotoxicity

Promacta® administration may cause hepatotoxicity. In the controlled clinical studies, one patient experienced Grade 4 (NCI Common Terminology Criteria for Adverse Events [NCI CTCAE] toxicity scale) elevations in serum liver test values during therapy with Promacta®, worsening of underlying cardiopulmonary disease and death. No patients in the placebo group experienced Grade 4 liver test abnormalities. Overall, serum liver test abnormalities (predominantly Grade 2 or less in severity) were reported in 10% and 8% of the Promacta® and placebo groups, respectively. In the controlled studies, two patients (1%) treated with Promacta® and two patients in the placebo group (3%) discontinued treatment due to hepatobiliary laboratory abnormalities. Seven of the patients treated with Promacta® in the controlled studies with hepatobiliary laboratory abnormalities were re-exposed to Promacta® in the extension study. Six of these patients again experienced liver test abnormalities (predominantly Grade 1) resulting in discontinuation of Promacta® in one patient. In the extension study, one additional patient had Promacta® discontinued due to liver test abnormalities (≦Grade 3).

Measure serum ALT, AST and bilirubin prior to initiation of Promacta®, every 2 weeks during the dose adjustment phase and monthly following establishment of a stable dose. If bilirubin is elevated, perform fractionation. Evaluate abnormal serum liver tests with repeat testing within 3 to 5 days. If the abnormalities are confirmed, monitor serum liver tests weekly until the abnormality(ies) resolve, stabilize, or return to baseline levels. Discontinue Promacta® if ALT levels increase to ≧3× the upper limit of normal (ULN) and are:
progressive, or
persistent for ≧4 weeks, or
accompanied by increased direct bilirubin, or
accompanied by clinical symptoms of liver injury or evidence for hepatic decompensation.

Reinitiating treatment with PROMACTA® is not recommended. If the potential benefit for reinitiating PROMACTA® treatment is considered to outweigh the risk for hepatotoxicity, then cautiously reintroduce PROMACTA® and measure scrum liver tests weekly during the dose adjustment phase. If liver tests abnormalities persist, worsen or recur, then permanently discontinue PROMACTA®.

Exercise caution when administering Promacta® to patients with hepatic disease. Use a lower starting dose of Promacta® in patients with moderate to severe hepatic disease and monitor closely [see Dosage and Administration (2.1)].

### 5.2 Bone Marrow Reticulin Formation and Risk for Bone Marrow Fibrosis

Promacta® is a thrombopoietin (TPO) receptor agonist and TPO-receptor agonists increase the risk for development or progression of reticulin fiber deposition within the bone marrow. In the extension study, seven patients had reticulin fiber deposition reported in bone marrow biopsies, including two patients who also had collagen fiber deposition. The fiber deposition was not associated with cytopenias and did not necessitate discontinuation of Promacta®. However, clinical studies have not excluded a risk of bone marrow fibrosis with cytopenias.

Prior to initiation of Promacta®, examine the peripheral blood smear closely to establish a baseline level of cellular morphologic abnormalities. Following identification of a stable dose of Promacta®, examine peripheral blood smears and CBCs monthly for new or worsening morphological abnormalities (e.g., teardrop and nucleated red blood cells, immature white blood cells) or cytopenia(s). If the patient develops new or worsening morphological abnormalities or cytopenia(s), discontinue treatment with Promacta® and consider a bone marrow biopsy, including staining for fibrosis.

5.3 Worsened Thrombocytopenia and Hemorrhage Risk after Cessation of Promacta®

Discontinuation of Promacta® may result in thrombocytopenia of greater severity than was present prior to therapy with Promacta®. This worsened thrombocytopenia may increase the patient's risk of bleeding, particularly if Promacta® is discontinued while the patient is on anticoagulants or antiplatelet agents. In the controlled clinical studies, transient decreases in platelet counts to levels lower than baseline were observed following discontinuation of treatment in 10% and 6% of the Promacta® and placebo groups, respectively. Serious hemorrhagic events requiring the use of supportive ITP medications occurred in 3 severely thrombocytopenic patients within one month following the discontinuation of Promacta®; none were reported among the placebo group.

Following discontinuation of Promacta®, obtain weekly CBCs, including platelet counts for at least 4 weeks and consider alternative treatments for worsening thrombocytopenia, according to current treatment guidelines [see Adverse Reactions (6.1)].

### 5.4 Thrombotic/Thromboembolic Complications

Thrombotic/thromboembolic complications may result from excessive increases in platelet counts. Excessive doses of Promacta® or medication errors that result in excessive doses of Promacta® may increase platelet counts to a level that produces thrombotic/thromboembolic complications. In the controlled clinical studies, one thrombotic/thromboembolic complication was reported within the groups that received Promacta® and none within the placebo groups. Seven patients experienced thrombotic/thromboembolic complications in the extension study. Use caution when administering Promacta® to patients with known risk factors for thromboembolism (e.g., Factor V Leiden, ATIII deficiency, antiphospholipid syndrome, etc). To minimize the risk for thrombotic/thromboembolic complications, do not use Promacta® in an attempt to normalize platelet counts. Follow the dose adjustment guidelines to achieve and maintain a platelet count of 50×10⁹/L [see Dosage and Administration (2.2)].

### 5.5 Malignancies and Progression of Malignancies

Promacta® stimulation of the TPO receptor on the surface of hematopoietic cells may increase the risk for hematologic malignancies. In the controlled clinical studies, patients were treated with Promacta® for a maximum of 6 weeks and during this period no hematologic malignancies were reported. One hematologic malignancy (non-Hodgkin's lymphoma) was reported in the extension study. Promacta® is not indicated for the treatment of thrombocytopenia due to causes of thrombocytopenia (e.g., myelodysplasia or chemotherapy) other than chronic ITP.

### 5.6 Laboratory Monitoring

Complete Blood Counts (CBCs):

Monitor CBCs, including platelet counts and peripheral blood smears, prior to initiation, throughout, and following discontinuation of therapy with Promacta®. Prior to the initiation of Promacta®, examine the peripheral blood differential to establish the extent of red and white blood cell abnormalities. Obtain CBCs, including platelet counts and peripheral blood smears, weekly during the dose adjustment phase of therapy with Promacta® and then monthly following establishment of a stable dose of Promacta®. Obtain CBCs, including platelet counts, weekly for at least 4 weeks following discontinuation of Promacta® [see Dosage and Administration (2.1) and Warnings and Precautions (5.1, 5.4)].

Liver Tests:

Monitor serum liver tests (ALT, AST, and bilirubin) prior to initiation of Promacta®, every 2 weeks during the dose adjustment phase and monthly following establishment of a stable dose. If bilirubin is elevated, perform fractionation. If abnormal levels are detected, repeat the tests within 3 to 5 days. If the abnormalities are confirmed, monitor serum liver tests weekly until the abnormality(ies) resolve, stabilize, or return to baseline levels. Discontinue Promacta® for the development of important liver test abnormalities [see Warnings and Precautions (5.1)].

### 5.7 Cataracts

In the controlled clinical studies, cataracts developed or worsened in five (5%) patients who received 50 mg Promacta® daily and two (3%) placebo-group patients. In the extension study, cataracts developed or worsened in 4% of patients who underwent ocular examination prior to therapy with Promacta®. Cataracts were observed in toxicology studies of eltrombopag in rodents [see Nonclinical Toxicology (13.2)]. Perform a baseline ocular examination prior to administration of Promacta® and, during therapy with Promacta®, regularly monitor patients for signs and symptoms of cataracts.

### 5.8 Promacta® Distribution Program

Promacta® is available only through a restricted distribution program called PROMACTA® CARES. Under PROMACTA® CARES, only prescribers, pharmacies, and patients registered with the program are able to prescribe, dispense, and receive Promacta®. This program provides educational materials and a mechanism for the proper use of Promacta®. To enroll in PROMACTA® CARES, call 1-877-9-PROMACTA®. Prescribers and patients are required to understand the risks of therapy with Promacta®. Prescribers are required to understand the information in the prescribing information and be able to:
Educate patients on the benefits and risks of treatment with Promacta®, ensure that the patient receives the Medication Guide, instruct them to read it, and encourage them to ask questions when considering Promacta®. Patients may be educated by the enrolled prescriber or a healthcare provider under that prescriber's direction.
Review the PROMACTA® CARES Prescriber Enrollment Forms, sign the form, and return the form according to Promacta® CARES Program instructions.
As part of the initial prescription process for Promacta®, obtain the patient's signature on the Patient Enrollment and Consent form, sign it, place the original signed form in the patient's medical record, send a copy to PROMACTA® CARES, and give a copy to the patient.
Report any serious adverse events associated with the use of Promacta® to PROMACTA® CARES Call Center at 1-877-9-PROMACTA® or to the FDA's MedWatch Program at 1-800-FDA-1088.
Report serious adverse events observed in patients receiving Promacta®, including events actively solicited at 6-month intervals.

### 6 ADVERSE REACTIONS

### 6.1 Clinical Trials Experience

In clinical studies, hemorrhage was the most common serious adverse reaction and most hemorrhagic reactions followed discontinuation of Promacta®. Other serious adverse reactions included liver test abnormalities and thrombotic/thromboembolic complications [see Warnings and Precautions (5.1, 5.2)].

The data described below reflect Promacta® exposure to 313 patients with chronic ITP aged 18 to 85, of whom 65% were female. Promacta® was studied in 2 randomized, placebo controlled studies in which patients received the drug for no more than 6 weeks. Promacta® was also studied in an open label single arm study in which patients received the drug over an extended period of time. Overall, Promacta® was administered to 81 patients for at least 6 months and 39 patients for at least 1 year.

Because clinical trials are conducted under widely varying conditions, adverse reaction rates observed in the clinical trials of a drug cannot be directly compared to rates in the clinical trials of another drug and may not reflect the rates observed in practice.

Table 2 presents the most common adverse drug reactions (experienced by more than 1 patient receiving Promacta®) from the placebo-controlled studies, with a higher incidence in Promacta® versus placebo.

TABLE 2
Adverse Reactions Identified in Two Placebo-Controlled Studies

| | Promacta ® 50 mg | Placebo |
|---|---|---|
| Preferred Term | n = 106% | n = 67% |
| Nausea | 6 | 4 |
| Vomiting | 4 | 3 |
| Menorrhagia | 4 | 1 |
| Myalgia | 3 | 1 |
| Paresthesia | 3 | 1 |
| Cataract | 3 | 1 |
| Dyspepsia | 2 | 0 |
| Ecchymosis | 2 | 1 |
| Thrombocytopenia | 2 | 0 |
| Increased ALT | 2 | 0 |
| Increased AST | 2 | 0 |
| Conjunctival hemorrhage | 2 | 1 |

Among 207 patients with chronic ITP who received Promacta® in the single arm extended study, the adverse reactions occurred in a pattern similar to those reported in the placebo-controlled studies.

### 7 Drug Interactions

### 7.1 Cytochrome P450

In vitro studies demonstrate that CYP1A2 and CYP2C8 are involved in the oxidative metabolism of eltrombopag. The significance of coadministration of Promacta® with 1) moderate or strong inhibitors of CYP 1A2 (e.g., ciprofloxacin, fluvoxamine) and CYP 2C8 (e.g., gemfibrozil, trimethoprim); 2) inducers of CYP 1A2 (e.g., tobacco, omeprazole) and CYP 2C8 (e.g., rifampin); or 3) other substrates of these CYP enzymes on the systemic exposure of Promacta® has not been established in clinical studies. Monitor patients for signs and symptoms of excessive eltrombopag exposure when Promacta® is administered concomitantly with these moderate or strong inhibitors of CYP1A2 or CYP2C8.

### 7.2 Transporters

In vitro studies demonstrate that eltrombopag is an inhibitor of the organic anion transporting polypeptide OATP1B1 and can increase the systemic exposure of other drugs that are substrates of this transporter (e.g., benzylpenicillin, atorvastatin, fluvastatin, pravastatin, rosuvastatin, methotrexate, nateglinide, repaglinide, rifampin). In a clinical study of healthy adult subjects administration of a single dose of rosuvastatin following repeated daily Promacta® dosing increased plasma rosuvastatin AUC_{0-∞} by 55% and Cₘₐₓ by 103% [see Clinical Pharmacology (12.3)].

Use caution when concomitantly administering Promacta® and drugs that are substrates of OATP1B1. Monitor patients closely for signs and symptoms of excessive exposure to the drugs that are substrates of OATP1B1 and consider reduction of the dose of these drugs. In clinical trials with eltrombopag, a dose reduction of rosuvastatin by 50% was recommended for coadministration with eltrombopag.

### 7.3 UDP-Glucuronosyltransferases (UGTs)

In vitro studies demonstrate that eltrombopag is an inhibitor of UGT1A1, UGT1A3, UGT1A4, UGT1A6, UGT1A9, UGT2B7, and UGT2B15, enzymes involved in the metabolism of multiple drugs, such as acetaminophen, narcotics, and nonsteroidal anti-inflammatory drugs (NSAIDs). The significance of this inhibition on the potential for increased systemic exposure of drugs that are substrates of these UGTs following coadministration with Promacta® has not been evaluated in clinical studies. Monitor patients closely for signs or symptoms of excessive exposure to these drugs when concomitantly administered with Promacta®.

In vitro studies demonstrate that UGT1A1 and UGT1A3 are responsible for the glucuronidation of Promacta®. The significance of coadministration of Promacta® with moderate or strong inhibitors or inducers on the systemic exposure of Promacta® has not been evaluated in clinical studies. Monitor patients closely for signs or symptoms of excessive exposure to Promacta® when concomitantly administered with these moderate or strong inhibitors of UGT1A1 or UGT1A3.

### 7.4 Polyvalent Cations (Chelation)

Eltrombopag chelates polyvalent cations (such as iron, calcium, aluminum, magnesium, selenium, and zinc) in foods, mineral supplements, and antacids. In a clinical study, administration of Promacta® with a polyvalent cation-containing antacid (1,524 mg aluminum hydroxide, 1,425 mg magnesium carbonate, and sodium alginate) decreased plasma eltrombopag systemic exposure by approximately 70% [see Clinical Pharmacology (12.3)].

Promacta® must not be taken within 4 hours of any medications or products containing polyvalent cations such as antacids, dairy products, and mineral supplements to avoid significant reduction in Promacta® absorption due to chelation [see Dosage and Administration (2)].

### 8 Use in Specific Populations

### 8.1 Pregnancy

### Pregnancy Category C

There are no adequate and well-controlled studies of eltrombopag use in pregnancy. In animal reproduction and developmental toxicity studies, there was evidence of embryolethality and reduced fetal weights at maternally toxic doses. Promacta® should be used in pregnancy only if the potential benefit justifies the potential risk to the fetus.

Pregnancy Registry:

A pregnancy registry has been established to collect information about the effects of Promacta® during pregnancy. Physicians are encouraged to register pregnant patients, or pregnant women may enroll themselves in the Promacta® pregnancy registry by calling 1-888-825-5249.

In an early embryonic development study, female rats received eltrombopag at doses of 0.8, 2, and 7 times the human clinical exposure (based on AUC). Increased pre- and post-implantation loss and reduced fetal weight were observed at the highest dose which also caused maternal toxicity.

In an embryofetal development study, pregnant rats received eltrombopag at doses of 0.8, 2, and 7 times the human clinical exposure (based on AUC). Decreased fetal weights and a slight increase in the presence of cervical ribs were observed at the highest dose which also caused maternal toxicity. However, no evidence of major structural malformations was observed. In an embryofetal development study in pregnant rabbits treated with oral eltrombopag doses of 0.1, 0.3, and 0.6 times the human clinical exposure (based on AUC) no evidence of fetotoxicity, embryolethality, or teratogenicity was observed.

In a pre- and post-natal developmental toxicity study in pregnant rats (F0), no adverse effects on maternal reproductive function or on the development of the offspring (F1) were observed at doses up to 2 times the human clinical exposure (based on AUC). Eltrombopag was detected in the plasma of offspring (F1). The plasma concentrations in pups increased with dose (0.8 and 2 times the human clinical exposure based on AUC) following administration of drug to the F0 dams.

### 8.3 Nursing Mothers

It is not known whether eltrombopag is excreted in human milk. Because many drugs are excreted in human milk and because of the potential for serious adverse reactions in nursing infants from Promacta®, a decision should be made whether to discontinue nursing or to discontinue Promacta® taking into account the importance of Promacta® to the mother and the known benefits of nursing.

### 8.4 Pediatric Use

The safety and efficacy of Promacta® in pediatric patients have not been established.

### 8.5 Geriatric Use

Of the 106 patients in 2 randomized clinical studies of Promacta® 50 mg dose, 22% were 65 years of age and older, and 9% were 75 years of age and older. No overall differences in safety or efficacy have been observed between older and younger patients in the placebo-controlled studies, but greater sensitivity of some older individuals cannot be ruled out. In general, dose adjustment for an elderly patient should be cautious, reflecting the greater frequency of decreased hepatic, renal, or cardiac function, and of concomitant disease or other drug therapy.

### 8.6 Hepatic Impairment

The disposition of Promacta® was compared in patients with hepatic impairment to subjects with normal hepatic function. Apparent clearance of Promacta® was reduced by approximately 50% in patients with moderate and severe (as indicated by the Child-Pugh method) hepatic impairment. In this clinical study that did not evaluate protein binding effects, the half-life of Promacta® was prolonged 2-fold in patients with moderate and severe hepatic impairment.

For patients with moderate and severe hepatic impairment, initiate Promacta® at a reduced dose of 25 mg once daily [see Dosage and Administration (2.1) and Warnings and Precautions (5.1)].

### 8.7 Renal Impairment

The safety and efficacy of Promacta® in patients with varying degrees of renal function have not been established. Closely monitor patients with impaired renal function when administering Promacta®.

### 10 Overdosage

In the event of overdose, platelet counts may increase excessively and result in thrombotic/thromboembolic complications. In case of an overdose, consider oral administration of a metal cation-containing preparation, such as calcium, aluminum, or magnesium preparations to chelate eltrombopag and thus limit absorption. Closely monitor platelet counts. Reinitiate treatment with Promacta® in accordance with dosing and administration recommendations [see Dosage and Administration (2.2)].

In one report, a subject ingested 5,000 mg of Promacta® and was treated with gastric lavage, oral lactulose, intravenous fluids, omeprazole, atropine, furosemide, calcium, dexamethasone, and plasmapheresis. The patient's platelet count increased to a maximum of 929×10⁹/L at 13 days following the ingestion. The patient also experienced rash, bradycardia, ALT/AST elevations, and fatigue. The abnormal platelet count and liver test abnormalities persisted for 3 weeks. After 2 months follow-up, all events had resolved without sequelae.

Hemodialysis is not expected to enhance the elimination of Promacta® because eltrombopag is not significantly renally excreted and is highly bound to plasma proteins.

### 11 Description

Promacta® (eltrombopag) Tablets contain cltrombopag olamine, a small molecule thrombopoietin (TPO) receptor agonist for oral administration. Eltrombopag interacts with the transmembrane domain of the TPO receptor (also known as cMp1) leading to increased platelet production. Each tablet contains eltrombopag olamine in the amount equivalent to 25 mg or 50 mg of eltrombopag free acid.

Eltrombopag olamine is a biphenyl hydrazone. The chemical name for eltrombopag olamine is 3′-{(2Z)-2-[1-(3,4-dimethylphenyl)-3-methyl-5-oxo-1,5-dihydro-4H-pyrazol-4-ylidene]hydrazino}-2′-hydroxy-3-biphenylcarboxylic acid-2-aminoethanol (1:2). It has the molecular formula C₂₅H₂₂N₄O₄.2(C₂H₇NO). The molecular weight is 564.65 for eltrombopag olamine and 442.5 for eltrombopag free acid. Eltrombopag olamine has the following structural formula:

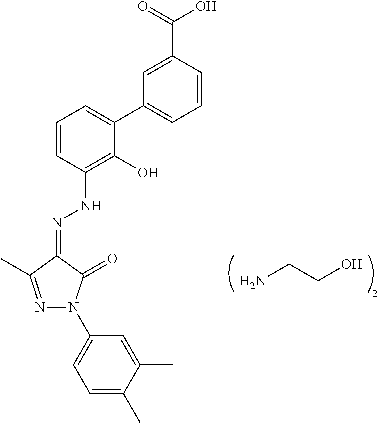

Eltrombopag olamine is practically insoluble in aqueous buffer across a pH range of 1 to 7.4, and is sparingly soluble in water.

The inactive ingredients of Promacta® are: Tablet Core: magnesium stearate, mannitol, microcrystalline cellulose, povidone, and sodium starch glycolate. Coating: hypromellose, polyethylene glycol 400, titanium dioxide, and FD&C Yellow No. 6 aluminum lake (25 mg tablet) or FD&C Blue No. 2 aluminum lake (50 mg tablet).

### 12 Clinical Pharmacology

### 12.1 Mechanism of Action

Eltrombopag is an orally bioavailable, small-molecule TPO-receptor agonist that interacts with the transmembrane domain of the human TPO-receptor and initiates signaling cascades that induce proliferation and differentiation of megakaryocytes from bone marrow progenitor cells.

### 12.2 Pharmacodynamics

### ECG Effects:

There is no indication of a QT/QTc prolonging effect of Promacta® in doses up to 150 mg daily for 5 days. The effects of Promacta® at doses up to 150 mg daily for 5 days (supratherapeutic doses) on the QT/QTc interval was evaluated in a double-blind, randomized, placebo- and positive-controlled (moxifloxacin 400 mg, single oral dose) crossover trial in healthy adult subjects. Assay sensitivity was confirmed by significant QTc prolongation by moxifloxacin.

### 12.3 Pharmacokinetics

A population pharmacokinetic model analysis suggests that the pharmacokinetic profile for eltrombopag following oral administration is best described by a 2-compartment model. Based on this model, the estimated exposures of eltrombopag after administration to patients with ITP are shown in Table 4.

TABLE 4
Geometric Mean (95% Confidence Intervals) of Steady-State
Plasma Eltrombopag Pharmacokinetic Parameters in Adults
With Idiopathic Thrombocytopenic Purpura

| | AUC_{(0-τ)} |
|---|---|
| Regimen of Promacta ® | (mcg · hr/mL) |
| 50 mg once daily (N = 34) | 91.9 |
| | (73.6, 115) |
| 75 mg once daily (N = 26) | 146 |
| | (122, 176) |

### Absorption:

Eltrombopag is absorbed with a peak concentration occurring 2 to 6 hours after oral administration. Based on urinary excretion and biotransformation products eliminated in feces, the oral absorption of drug-related material following administration of a single 75 mg solution dose was estimated to be at least 52%.

In a clinical study, administration of a single 75 mg-dose of Promacta® with a polyvalent cation-containing antacid (1,524 mg aluminum hydroxide, 1,425 mg magnesium carbonate, and sodium alginate) decreased plasma eltrombopag AUC_{0-∞} and Cₘₐₓ by 70%. The contribution of sodium alginate to this interaction is not known [see Drug Interactions (7.4)].

An open-label, randomized, crossover study was conducted to assess the effect of food on the bioavailability of eltrombopag. A standard high-fat breakfast significantly decreased plasma cltrombopag AUC_{0-∞} by approximately 59% and Cₘₐₓ by 65% and delayed tₘₐₓ by 1 hour. The calcium content of this meal may have also contributed to this decrease in exposure.

### Distribution:

The concentration of eltrombopag in blood cells is approximately 50-79% of plasma concentrations based on a radiolabel study. In vitro studies suggest that eltrombopag is highly bound to human plasma proteins (>99%). Eltrombopag is not a substrate for p-glycoprotein (Pgp) or OATP1B1.

### Metabolism:

Absorbed eltrombopag is extensively metabolized, predominately through pathways including cleavage, oxidation, and conjugation with glucuronic acid, glutathione, or cysteine. In a human radiolabel study, eltrombopag accounted for approximately 64% of plasma radiocarbon AUC_{0-∞}. Metabolites due to glucuronidation and oxidation were also detected. In vitro studies suggest that CYP 1A2 and 2C8 are responsible for the oxidative metabolism of eltrombopag. UGT1A1 and UGT1A3 are responsible for the glucuronidation of eltrombopag.

### Elimination:

The predominant route of eltrombopag excretion is via feces (59%), and 31% of the dose is found in the urine. Unchanged eltrombopag in feces accounts for approximately 20% of the dose; unchanged eltrombopag is not detectable in urine. The plasma elimination half-life of cltrombopag is approximately 21 to 32 hours in healthy subjects and 26-35 hours in ITP patients.

### Race:

Based on both non-compartment analysis and population pharmacokinetic analysis, plasma eltrombopag exposure was approximately 70% higher in some Asian subjects of Japanese, Chinese, Taiwanese, and Korean ancestry (i.e., East Asian) with ITP as compared to non-Asian subjects who were predominantly Caucasian [see Dosage and Administration (2.1)]. In addition, the pharmacodynamic (PD) response to eltrombopag was qualitatively similar in the Asian subjects, but the absolute PD response was somewhat greater.

An approximately 40% higher systemic eltrombopag exposure in healthy African-American subjects was noted in at least one clinical pharmacology study. The effect of African-American ethnicity on exposure and related safety and efficacy of eltrombopag has not been established.

### Gender:

Results from a population pharmacokinetic model suggest that males have a 27% greater apparent eltrombopag clearance than females, after adjustment for the body weight difference.

### Hepatic Impairment:

Plasma eltrombopag pharmacokinetics in subjects with mild, moderate, and severe hepatic impairment compared to healthy subjects was investigated following administration of a single 50 mg dose of eltrombopag. The degree of hepatic impairment was based on Child-Pugh score. Plasma eltrombopag AUC_{0-∞} was 41% higher in subjects with mild hepatic impairment, and 80% to 93% higher in subjects with moderate to severe hepatic impairment compared with healthy subjects. A corresponding reduction in apparent clearance was also reported. The impact of hepatic impairment was highly variable between subjects. Unbound eltrombopag (active) concentrations for this highly protein bound drug was not measured [see Dosage and Administration (2.1)] and Use in Specific Populations (8.6)].

### Renal Impairment:

The pharmacokinetics of eltrombopag have not been established in patients with renal impairment [see Use in Specific Populations (8.7)].

### Drug Interactions:

### Cytochrome P450:

In vitro studies report that cltrombopag is an inhibitor of CYP2C8 and CYP2C9 as measured using paclitaxel and diclofenac as the probe substrates. A clinical study where Promacta® 75 mg once daily was administered for 7 days to 24 healthy male subjects did not show inhibition or induction of the metabolism of a combination of probe substrates for CYP 1A2 (caffeine), CYP2C19 (omeprazole), CYP2C9 (flurbiprofen), or CYP3A4 (midazolam) in humans. Probe substrates for CYP2C8 were not evaluated in this study.

In vitro studies suggest that CYP 1 A2 and 2C8 are responsible for oxidative metabolism of cltrombopag. Clinical studies evaluating the effect of strong inducers or inhibitors of these CYP enzymes responsible for the metabolism of eltrombopag have not been conducted.

### Transporters:

In vitro studies demonstrated that eltrombopag is an inhibitor of the OATP1B1. Administration of 75 mg of Promacta® once daily for 5 days with a single 10 mg-dose of the OATP1B1 substrate, rosuvastatin, to 39 healthy adult subjects increased plasma rosuvastatin AUC_{0-∞} by 55% and Cₘₐₓ by 103% [see Drug Interactions (7.2)].

### UDP-Glucuronosyltransferases (UGTs): See Drug Interactions (7.3).

### 13 Nonclinical Toxicology

### 13.1 Carcinogenesis, Mutagenesis, Impairment of Fertility

Eltrombopag does not stimulate platelet production in rats, mice, or dogs because of unique TPO-receptor specificity. Data from these animals do not fully model effects in humans.

Eltrombopag was not carcinogenic in mice at doses up to 75 mg/kg/day or in rats at doses up to 40 mg/kg/day (exposures up to 4 and 5 times the human clinical exposure based on AUC, respectively).

Eltrombopag was not mutagenic or elastogenic in a bacterial mutation assay or in 2 in vivo assays in rats (micronucleus and unscheduled DNA synthesis, 11 times the human clinical exposure based on Cₘₐₓ). In the in vitro mouse lymphoma assay, eltrombopag was marginally positive (<3-fold increase in mutation frequency).

Eltrombopag did not affect female fertility in rats at doses up to 20 mg/kg/day (2 times the human clinical exposure based on AUC). Eltrombopag did not affect male fertility in rats at doses up to 40 mg/kg/day, the highest dose tested (5 times the human clinical exposure based on AUC).

### 13.2 Animal Pharmacology/Toxicology

Eltrombopag is phototoxic and photoelastogenic in vitro. In vitro photoelastogenic effects were observed only at cytotoxic drug concentrations (≧15 mcg/mL) and at UV light exposure intensity (30 MED, minimal erythematous dose). No evidence of in vitro photoelastogenicity was observed at higher drug concentrations (up to 58.4 mcg/mL) and UV light exposure of 15 MED. There was no evidence of in vivo cutaneous phototoxicity in mice, photo-ocular toxicity in rats or photo-ocular toxicity in mice at exposures up to 11, 6, and 7 times the human clinical exposure based on AUC, respectively.

Treatment-related cataracts were detected in rodents in a dose- and time-dependent manner. At 7 times the human clinical exposure based on AUC, cataracts were observed in mice after 6 weeks and in rats after 28 weeks of dosing. At ≧5 times the human clinical exposure based on AUC, cataracts were observed in mice after 13 weeks and in rats after 39 weeks of dosing. Cataracts were not observed in dogs after 52 weeks of dosing (3 times the human clinical exposure based on AUC). The clinical relevance of these findings is unknown [see Warnings and Precautions (5.7)].

Renal tubular toxicity was observed in studies up to 14 days in duration in mice and rats at exposures that were generally associated with morbidity and mortality. Tubular toxicity was also observed in a 2-year oral carcinogenicity study in mice at doses of 25, 75, and 150 mg/kg/day. The exposure at the lowest dose was 1.4 times the human clinical exposure based on AUC. No similar effects were observed after 13 weeks at exposures greater than those associated with renal changes in the 2-year study, suggesting that this effect is both dose- and time-dependent. Renal tubular toxicity was not observed in rats in a 2-year carcinogenicity study or in dogs after 52 weeks at exposures 5 and 3 times the human clinical exposure based on AUC, respectively.

Eltrombopag produced hepatocellular hypertrophy in mice (7 times the human clinical exposure based on AUC), rats (5 times the human clinical exposure based on AUC), rabbits (1.4 times the human clinical exposure based on AUC), and dogs (4 times the human clinical exposure based on AUC) and hepatocellular vacuolation in rats (2 times the human clinical exposure based on AUC)

### 13.3 Reproductive and Developmental Toxicology

Eltrombopag was administered orally to pregnant rats in an embryofetal development study at 10, 20, or 60 mg/kg/day (0.8, 2, and 7 times the human clinical exposure, respectively, based on AUC). Decreases in maternal body weight gain and food consumption occurred in the 60 mg/kg/day dose group. At this maternally toxic dose, male and female fetal weights were significantly reduced (6% to 7%) and there was a slight increase in the presence of cervical ribs, a fetal variation.

In an embryofetal development study in mated female rabbits, eltrombopag was administered orally at 30, 80, or 150 mg/kg/day (0.1, 0.3, and 0.6 times the human clinical exposure, respectively, based on AUC). There was no evidence of fetotoxicity, embryolethality, or teratogenicity at any dose.

In a pre- and post-natal developmental toxicity study in pregnant rats (F0), no adverse effects on maternal reproductive function or on the development of the offspring (F1) were observed at doses up to 2 times the human clinical exposure (based on AUC). Eltrombopag was detected in the plasma of offspring (F1). The plasma concentrations in pups increased with dose (0.8 and 2 times the human clinical exposure based on AUC) following administration of drug to the F0 dams.

### 14 Clinical Studies

The efficacy and safety of Promacta® in adult patients with chronic ITP were evaluated in 2 randomized double-blind, placebo-controlled studies and in an open-label extension study.

### 14.1 Studies 1 and 2

In studies 1 and 2, patients who had completed at least one prior ITP therapy and who had a platelet count<30×10⁹/L were randomized to either daily placebo or Promacta® administered over a maximum treatment period of 6 weeks, followed by 6 weeks off therapy. During the studies, Promacta® or placebo were discontinued if the platelet count exceeded 200×10⁹/L. The primary efficacy endpoint was response rate, defined as a shift from a baseline platelet count of <30×10⁹/L to ≧50×10⁹/L at any time during the treatment period.

The median age of the patients was 50 years and 60% were female. Approximately 70% of the patients had received at least 2 prior ITP therapies (predominantly corticosteroids, immunoglobulins, rituximab, cytotoxic therapies, danazol, and azathioprine) and 40% of the patients had undergone splenectomy. The median baseline platelet counts (approximately 18×10⁹/L) were similar among all treatment groups.

Study 1 randomized 114 patients (2:1) to Promacta® 50 mg or placebo. Study 2 randomized 117 patients (1:1:1:1) among placebo or one of three dose regimens of Promacta®, 30 mg, 50 mg, or 75 mg each administered daily.

Table 5 shows the outcomes for the placebo groups and the groups of patients who received the 50 mg daily regimen of Promacta®.

TABLE 5
Studies 1 and 2 Platelet Count Response (≧50 × 10⁹/L) Rates

| | Promacta ® | |
|---|---|---|
| Study | 50 mg Daily | Placebo |
| 1 | 43/73 (59%)* | 6/37 (16%) |
| 2 | 19/27 (70%)* | 3/27 (11%) |

*p < 0.001 for Promacta ® versus placebo.

The platelet count response to Promacta® was similar among patients who had or had not undergone splenectomy. In general, increases in platelet counts were detected 1 week following initiation of Promacta® and the maximum response observed after 2 weeks of therapy. Within the placebo and 50 mg dose group of Promacta®, the study drug was discontinued due to an increase in platelet counts to >200×10⁹/L in 3% and 27% of the patients, respectively. The median duration of treatment with the 50 mg dose of Promacta® in Study 1 was 42 days and Study 2 was 43 days.

Of seven patients (three in the placebo group and four in the group that received Promacta®) who underwent hemostatic challenges, additional ITP medications were required in all placebo group patients and none of the patients treated with Promacta®. Surgical procedures accounted for most of the hemostatic challenges. Hemorrhage requiring transfusion occurred in one placebo group patient and no patients treated with Promacta®.

### 14.2 Extension Study

Patients who completed any prior clinical study with Promacta® were enrolled in an open label, single arm study in which attempts were made to decrease the dose or eliminate the need for any concomitant ITP medications. Promacta® was administered to 109 patients; 74 completed 3 months of treatment, 53 completed 6 months and three patients completed 1 year of therapy. The median baseline platelet count was 18×10⁹/L prior to administration of Promacta®. Median platelet counts at 3, 6, and 9 months on study were 74×10⁹/L, 67×10⁹/L, and 95×10⁹/L, respectively. The median daily dose of Promacta® following 6 months of therapy was 50 mg (n=53); the median daily dose was also 50 mg among patients with no change in the dose regimen of Promacta® over 2 months or more of therapy (n=45).

### 16 How Supplied/Storage and Handling

The 25 mg tablets are round, biconvex, orange, film-coated tablets debossed with GS NX3 and 25 on one side and are available in bottles of 30: NDC 0007-4640-13.

The 50 mg tablets are round, biconvex, blue, film-coated tablets debossed with GS UFU and 50 on one side and are available in bottles of 30: NDC 0007-4641-13.

Store at 25° C. (77° F.); excursions permitted to 15° to 30° C. (59° to 86° F.) [see USP Controlled Room Temperature].

### 17 Patient Counseling Information

See Medication Guide (17.2).

### 17.1 Information for Patients

Prior to treatment, patients should fully understand the risks and benefits of Promacta®. Inform patients that the risks associated with long-term administration of Promacta® are unknown and that they must enroll in PROMACTA® CARES, which provides for the proper use of Promacta® in ITP patients.

Inform patients of the following risks and considerations for Promacta®:
Therapy with Promacta® is administered to achieve and maintain a platelet count≧50×10⁹/L as necessary to reduce the risk for bleeding; Promacta® is not used to normalize platelet counts.
Therapy with PROMACTA® may be associated with hepatobiliary laboratory abnormalities. Monitor serum liver tests (ALT, AST, and bilirubin) prior to initiation of PROMACTA®, every 2 weeks during the dose adjustment phase, and monthly following establishment of a stable dose. If bilirubin is elevated, perform fractionation.
Inform patients that they should report any of the following signs and symptoms of liver problems to their healthcare provider right away.
   yellowing of the skin or the whites of the eyes (jaundice),
   unusual darkening of the urine
   unusual tiredness,
   right upper stomach area pain.
Following discontinuation of Promacta®, thrombocytopenia and risk of bleeding may develop that is worse than that experienced prior to therapy with Promacta®, particularly if Promacta® is discontinued while the patient is on anticoagulants or antiplatelet agents.
Therapy with Promacta® increases the risk of reticulin fiber formation within the bone marrow, and further fiber formation may progress to marrow fibrosis. Detection of peripheral blood cell abnormalities may necessitate a bone marrow examination.
Too much Promacta® may result in excessive platelet counts and a risk for thrombotic/thromboembolic complications.
Promacta® stimulates certain bone marrow cells to make platelets and may increase the risk for progression of underlying MDS or hematological malignancies.
Platelet counts and CBCs, including peripheral blood smears, must be performed weekly until a stable dose of Promacta® has been achieved; thereafter, platelet counts and CBCs, including peripheral blood smears, must be performed monthly while taking Promacta®.
Patients must be closely monitored with weekly platelet counts and CBCs for at least 4 weeks following discontinuation of Promacta®.
Even during therapy with Promacta®, patients should continue to avoid situations or medications that may increase the risk for bleeding.
Patients must be advised to keep at least a 4 hour interval between Promacta® and foods, mineral supplements, and antacids which contain polyvalent cations such as iron, calcium, aluminum, magnesium, selenium, and zinc.

Promacta® is a registered trademark of GlaxoSmithKline.

The compound 3′-{N′-[1-(3,4-Dimethylphenyl)-3-methyl-5-oxo-1,5-dihydropyrazol-4-ylidene]hydrazino}-2′-hydroxybiphenyl-3-carboxylic acid is disclosed and claimed, along with pharmaceutically acceptable salts, hydrates, solvates and esters thereof, as being useful as an agonist of the TPO receptor, particularly in enhancing platelet production and particularly in the treatment of thrombocytopenia, in International Application No. PCT/US01/16863, having an International filing date of May 24, 2001; International Publication Number WO 01/89457 and an International Publication date of Nov. 29, 2001, the entire disclosure of which is hereby incorporated by reference. The compound 3′-{N′-[1-(3,4-Dimethylphenyl)-3-methyl-5-oxo-1,5-dihydropyrazol-4-ylidene]hydrazino}-2′-hydroxybiphenyl-3-carboxylic acid and pharmaceutically acceptable salts, hydrates, solvates and esters thereof, are prepared as described in International Application No. PCT/US01/16863. The bis-(monoethanolamine) salt of the compound is described in International Application No. PCT/US03/16255, having an International filing date of May 21, 2003; International Publication Number WO 03/098992 and an International Publication date of Dec. 4, 2003, the entire disclosure of which is hereby incorporated by reference. Formulations containing the compound are described in International Application No. PCT/US07/074918, having an International filing date of Aug. 1, 2007; the entire disclosure of which is hereby incorporated by reference.

Without further elaboration, it is believed that one skilled in the art can, using the preceding description, utilize the present invention to its fullest extent. The following Examples are, therefore, to be construed as merely illustrative and not a limitation of the scope of the present invention in any way.

### EXPERIMENTAL DETAILS

### Example 1

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| 3′-{N′-[1-(3,4-Dimethylphenyl)-3-methyl-5-oxo-1,5- | 25 mg |
| dihydropyrazol-4-ylidene]hydrazino}-2′-hydroxybiphenyl- | |
| 3-carboxylic acid bis-(monoethanolamine) | |
| Mannitol | 55 mg |
| Talc | 16 mg |
| Magnesium Stearate | 4 mg |

### Example 2

### Injectable Parenteral Composition

An injectable form for administering the present invention is produced by stirring 1.5% by weight of 3′-{N′-[1-(3,4-Dimethylphenyl)-3-methyl-5-oxo-1,5-dihydropyrazol-4-ylidene]hydrazino}-2′-hydroxybiphenyl-3-carboxylic acid bis-(monoethanolamine) in 10% by volume propylene glycol in water.

### Example 3

### Tablet Composition

The sucrose, calcium sulfate dihydrate and a non-peptide TPO agonist, as shown in Table II below, are mixed and granulated in the proportions shown with a 10% gelatin solution. The wet granules are screened, dried, mixed with the starch, talc and stearic acid, then screened and compressed into a tablet.

TABLE II

| INGREDIENTS | AMOUNTS |
|---|---|
| 3′-{N′-[1-(3,4-dimethylphenyl)-3-methyl-5-oxo-1,5- | 20 mg |
| dihydropyrazol-4-ylidene]hydrazino}-2′-hydroxybiphenyl- | |
| 3-carboxylic acid bis-(monoethanolamine) | |
| Microcrystalline cellulose | 30 mg |
| sucrose | 4 mg |
| starch | 2 mg |
| talc | 1 mg |
| stearic acid | 0.5 mg |

While the preferred embodiments of the invention are illustrated by the above, it is to be understood that the invention is not limited to the precise instructions herein disclosed and that the right to all modifications coming within the scope of the following claims is reserved.

What is claimed is:

1. A method of administering the compound: 3′-[(2Z)-[1-(3,4-dimethylphenyl)-1,5-dihydro-3-methyl-5-oxo-4H-pyrazol-4-ylidene]hydrazino)-2′-hydroxy-[1,1′-biphenyl]-3-carboxylic acid bis-(monoethanolamine), wherein the compound is administered once daily and each dose is administered more than four hours from the administration of a medicine or product that contains polyvalent cations.

2. A method according to claim 1 wherein the compound is administered in an amount selected from: 25 mg, 50 mg and 75 mg, based on the amount of free or unsalted compound.

3. A method according to claim 1 wherein the polyvalent cation is in a form selected from:
antacids, dairy products and mineral supplements.

4. A method according to claim 1 wherein the polyvalent cation is selected from: iron, calcium, aluminum, magnesium, selenium and zinc.

5. A method of administering the compound: 3′-[(2Z)-[1-(3,4-dimethylphenyl)-1,5-dihydro-3-methyl-5-oxo-4H-pyrazol-4-ylidene]hydrazino)-2′-hydroxy-[1,1′-biphenyl]-3-carboxylic acid bis-(monoethanolamine), wherein the compound is administered once daily for at least one week and each dose is administered more than four hours from the administration of a medicine or product that contains polyvalent cations.

6. A method according to claim 5 wherein the compound is administered in an amount selected from: 25 mg, 50 mg and 75 mg, based on the amount of free or unsalted compound.

7. A method according to claim 5 wherein the polyvalent cation is in a form selected from: antacids, dairy products and mineral supplements.

8. A method according to claim 5 wherein the polyvalent cation is selected from: iron, calcium, aluminum, magnesium, selenium and zinc.

9. A method of administering the compound: 3′-[(2Z)-[1-(3,4-dimethylphenyl)-1,5-dihydro-3-methyl-5-oxo-4H-pyrazol-4-ylidene]hydrazino)-2′-hydroxy-[1,1′-biphenyl]-3-carboxylic acid bis-(monoethanolamine), wherein the compound is administered once daily for at least four weeks and each dose is administered more than four hours from the administration of a medicine or product that contains polyvalent cations.

10. A method according to claim 9 wherein the compound is administered in an amount selected from: 25 mg, 50 mg and 75 mg, based on the amount of free or unsalted compound.

11. A method according to claim 9 wherein the polyvalent cation is in a form selected from: antacids, dairy products and mineral supplements.

12. A method according to claim 9 wherein the polyvalent cation is selected from: iron, calcium, aluminum, magnesium, selenium and zinc.
